(19) United States
(12) Patent Application Publication
(10) Pub. No.: US 2006/0144956 A1
(43) Pub. Date: Jul. 6, 2006

(54) Device for delivering an aromatic substance or fragrance

(76) Inventor: Stefan Ruetz, Munchen (DE)

Correspondence Address: SMITH-HILL AND BEDELL, P.C., 16100 NW CORNELL ROAD, SUITE 220, BEAVERTON, OR, 97006, US

(21) Appl. No.: 10/546,928

(22) PCT Filed: Feb. 12, 2004
(86) PCT No.: PCT/EP04/01325

§ 371 Date: Sep. 7, 2005

(30) Foreign Application Priority Data
Feb. 27, 2003 (DE) 103 08 619.6

(51) Int. Cl. A61L 9/04; A24F 25/00

(52) U.S. Cl. 239/34; 422/123; 422/5; 239/102.1

(57) ABSTRACT

The invention relates to a device for dispensing a substance, particularly a scent, comprising a tank for filling with the substance and a dispenser assigned to the tank, comprising means for delivering the substance into an environmental medium. The tank and its dispenser are configured as a unit closed off from the environmental medium after each dispensing to substantially prevent contact of the environmental medium with the substance contained in the tank.

The invention relates to a device for dispensing a substance, particularly a scent or fragrancy, comprising a tank for filling with the substance and a dispenser assigned to the tank. The invention also relates to a method for dispensing such a substance. KNOWN from EP 0 611 476 B1 is a method for enhancing the appreciation of sight and/or sound performances in cinema, theater or concert halls by delivering fragrancies to the audience, heightening the particularly scene or sound in timing therewith.

One such apparatus for discharging fragrancies is known, for example. from WO 00/78367 A1. This apparatus comprises an aroma store, a controller with a receiving module for controlling the aroma store, as well as a dispenser for generating and dispensing a cloud of fragrancy or aroma from the aroma store. The apparatus is devised as a miniaturized mobile unit for wearing on the body of the user or for arranging in the vicinity of the user.

In accordance with prior art, a variety of fragrancies are each housed an a storage chamber, a plurality of which are combined in a replaceable unit in the form of a cartridge. When a fragrancy in a storage chamber of a cartridge unit has been exhausted, the cartridge is replaced by a new one. So that there is no unwanted release of fragrancies, the receiving chambers are provided with closing means which are opened on dispensing the fragrancy.

It is likewise known from prior art to release fragrancies as a function of the temperature, i.e. the fragrancy not being released and diffused into the environment until a certain temperature is attained. Releasing the fragrancy in this way is intended to simultaneously solve the problem of preventing an unwanted escape of the fragrancy.

The invention is based on the object of providing a device for dispensing a substance, particularly a scent which is simple and cost-effective in production. It is furthermore an object of the invention to maintain the quality of the substance or scent for dispensing within a time window.

These objects are achieved by a device for dispensing a substance, particularly a scent as it reads from claim 1. Advantageous embodiments of the invention read from each of the sub-claims. As set forth in claim 13 a preferred method is proposed for dispensing a substance, particularly a scent.

The device in accordance with the invention is devised for dispensing an olfactory perceptible fragrancy or scent in an environmental medium, particularly air. As the substance for use in the device any substance is suitable which is capable of being transported in its components. Such a substance may be housed in the device in gaseous, liquid, pasty or gel form. It is just as possible that the substance is composed of a scent for dispensing and a vehicle medium such as, for example, a gas or a liquid which are mixed in the tank and dispensed together by the dispenser.

A tank accommodating the substance and a dispenser assigned to the tank form as regards the environmental medium a self-contained unit within which contact of the environmental medium with the substance in the tank is substantially prevented. The phrase “substantially preventing” in this context defines that although contact of individual atoms or molecules of the environmental medium with the substance is not absolutely excluded, the proportion thereof is negligably small. In other words, a device for dispensing a scent when used with air as the environmental medium is hermetically sealed from the air when contact of air with the housed scent in the tank substantially does not occur.

Since contact of the scent with oxygen existing in the environmental air diminishes the quality of the scent, the aspect in accordance with the invention that no exchange of environmental air and the substance occurs in the tank is achieved in that the quality of the scent is maintained unchanged over a lengthy period of time.

Apart from this, it is assured that the substance dispensed as the scent is not dispensed to the environmental medium when not wanted. Dispensing the substance occurs exclusively on actuation of the means of the dispenser provided for delivering the substance into the environmental medium.

The device in accordance with the invention is intended for use both personally and commercially, examples of some applications being: shopping, relaxation, meditation, video games, television, video, computer simulations, Internet, cinema, theater, exhibitions, to name but a few. Dimensioning the device and particularly the tank depends on the particularly application involved. For instance, a miniaturized device in the form of a fragrance chip is feasible as can be worn on the body of the user. The unit comprising the fillable tank and dispenser can be manufactured cost-effectively in a single production process.

In one advantageous embodiment of the device the tank or a portion connected thereto comprises a sealable filling port via which the tank can be evacuated and filled with a substance. Connectable to the filling port is a filler by means of which the interior of the tank is emptied of any environmental medium or substance therein, before the tank is filled with a new substance.

In one advantageous aspect of the invention it is provided for that the tank comprises at least in part a deformable wall which during evacuation comes into contact with a non-deformable wall of the tank to thus ensure that when evacuating the tank no negative pressure materializes in its interior relative to the environmental medium. Important in this respect is that the material for the deformable tank wall and the shape of the tank are selected so that a complete collapse of the tank is achieved in enabling the tank to be totally evacuated without voids remaining in the interior of the tank with the result that no remainders whatsoever remain in the tank after evacuation. Accordingly, the material to be used for the deformable tank wall is one which can come into contact with the non-deformable wall of the tank where possible free of an wrinkles or folds. Advantageous materials for use as the deformable tank wall are suitable plastics films. After evacuation, the tank can be filled with a new substance, the volume of the tank increasing due to the deformable tank wall as a function of the volume of the filled substance. The substance may also be filled into the tank with a gas or liquid compatible with the substance as a vehicle therefor. It is just as possible to provide a tank made exclusively of a deformable, i.e. flexible film, it in any case needing to be assured that in totally evacuating the tank no voids remain in the tank.

In another advantageous embodiment of the device in accordance with the invention it is provided for that the means for delivering the substance comprise a micropump. The micropump is configured to ensure that sufficient protection against unwanted escape of the substance from the tank is provided whilst preventing ingress of the environmental medium into the tank. Such a micropump may be for example a membrane, rotary flexible or piston type pump.

A preferred micropump is a peristaltic pump comprising an inlet valve, an outlet valve and at least one pumping chamber between the inlet valve and outlet valve. Assigned to each inlet valve, the outlet valve and the pumping chamber(s) is a deformable membrane on which actuators are arranged. By a suitable arrangement of the valves relative to the pumping chamber(s) a sole membrane may be provided extending over both valves and the pumping chamber(s). By means of the actuators the membrane can be deformed in thus opening or closing the inlet valve and outlet valve and/or a desired pressure created in the at least one pumping chamber. Advantageously, the valves are closed when the membrane is relaxed, i.e. when the actuators are not activated.

Actuating the peristaltic pump delivers the substance by a time-controlled actuation of the actuators as follows: with the outlet valve closed, the inlet valve is opened by activating the inlet valve actuator and roughly at the same time by activating the pumping chamber actuator to produce a negative pressure in the at least one pumping chamber so that substance is suctioned from the tank via the inlet valve into the pumping chamber. After closing the inlet valve the outlet valve is opened by activating the actuator asigned to the outlet valve and by activating the actuator assigned to the pumping chamber creates a pressure therein with which the substance is dispensed into the environmental medium. Higher differences in pressure are achievable by using a plurality of pumping chambers in series.

In this arrangement the actuators are activated such that at no point in time is a connection created from the interior of the tank to the environmental medium, even during actuation of the pump, so that at no point in time can the environmental medium gain access to the tank. This is achieved by the two valves not being opened at the same time. The advantage in this embodiment is that such a peristaltic pump can be produced extremely miniaturized for reliable operation.

Advantageously, electrically activatable piezo elements can be provided as actuators for the inlet valve and outlet valve and the at least one pumping chamber, it being deemed advantageous at the same time to make use of electrically activatable and thermally operating actuators.

It is furthermore deemed to be of advantage that the actuator membrane assigned to the inlet valve and outlet valve and the pumping chamber(s) extends into the region of the outlet passage for delivering the substance where it is configured with an optionally small orifice. With the aid of the thus configured orifice, forming and dispensing a compact droplet of the substance is assured, it being likewise possible to configure the orifice as an aerosol nozzle.

Instead of the membrane orifice any other nozzle for delivering the substance as an aerosol can be employed, for example an ultrasonic nebulizer.

Another advantageous aspect of the device is characterized by a contactor being provided by means of which the device for dispensing the substance can be connected to a controller for actuating the dispenser. The controller in this arrangement may be a component of the device in accordance with the invention. A controller, expediently with a receiving module for external activation by a signalling unit or clock permits dispensing a scent in response to a signal output by the controller. The control signal may comprise information as to the nature and/or composition of the fragrancy as well as information as to the duration of fragrancy delivery, for example. Furthermore, the fragrancy control signals may preferably contain information as to the intensity of the fragrancy, start and end of fragrancy delivery. As such signals coded film tracks or event-coupled signal transmitters may be employed in thus making it possible to use the device for dispensing a scent in synchronism with a visual performance such as, for example, a motion picture so that a synchronizing signal is sent by the controller to the device in certain sequences of the motion picture to dispense a fragrancy in harmony with the sequence.

In yet another aspect of the invention it is provided for that the dispenser comprises a hot plate inserted in the delivery path of the substance so that evaporation of the substance is assisted by heating or on the substance impacting the hot plate. The resulting better convection of the substance into the environmental medium achieves a faster olfactory appreciation of the dispensed scent. As aforementioned, advantageously a droplet of the substance is formed by the membrane orifice so that the droplet fully impacts the hot plate.

In still another advantageous aspect of the device a blower is provided to create a stream of air delivering the dispensed substance, it being important in this arrangement that the substance is dispensed without a gas vehicle, i.e. no gas passing through the tank to dispense the substance, In other words, the blower stream simply supports the natural convection of the environmental medium for olfactory appreciation.

In connection with the blower in another advantageous embodiment there is provided a restrictor or jetting device by means of which tank air stream generated by the blower is split into an inner gas stream carrying the substance to be dispensed and an outer gas stream surrounding the inner gas stream. The outer gas stream forms an insulation from the air stream carrying the substance in thus making it possible to deliver the dispensed substance projected into the environmental medium over a large distance without a user being aware of this.

In still another advantageous aspect of the invention a plurality of units each comprising a tank and dispenser are combined into a single device, i.e. by creating a cartridge in which a plurality of tanks each with a different substance, particularly different scents, are combined. The cartridge may at the same time comprise a contacter and/or a controller for actuating all dispensers combined in the cartridge, as a result of which individual fragrancies can be dispensed by activating the individual dispenser means combined in the cartridge.

Advantageously, the device or the cartridges include means for identifying the substance held in each tank. Such means may comprise an electronic memory for contacting via the contacter so that a data link is created for communicating the identification data of the substances from the device or cartridge to the controller.

The elements of the device in contact with the substance are made of a material which is immune to reacting with the received substances, one such suitable material being, for example, polyetheretherketon (PEEK).

The invention will now be detained by way of an example embodiment with respect to the attached drawings in which:

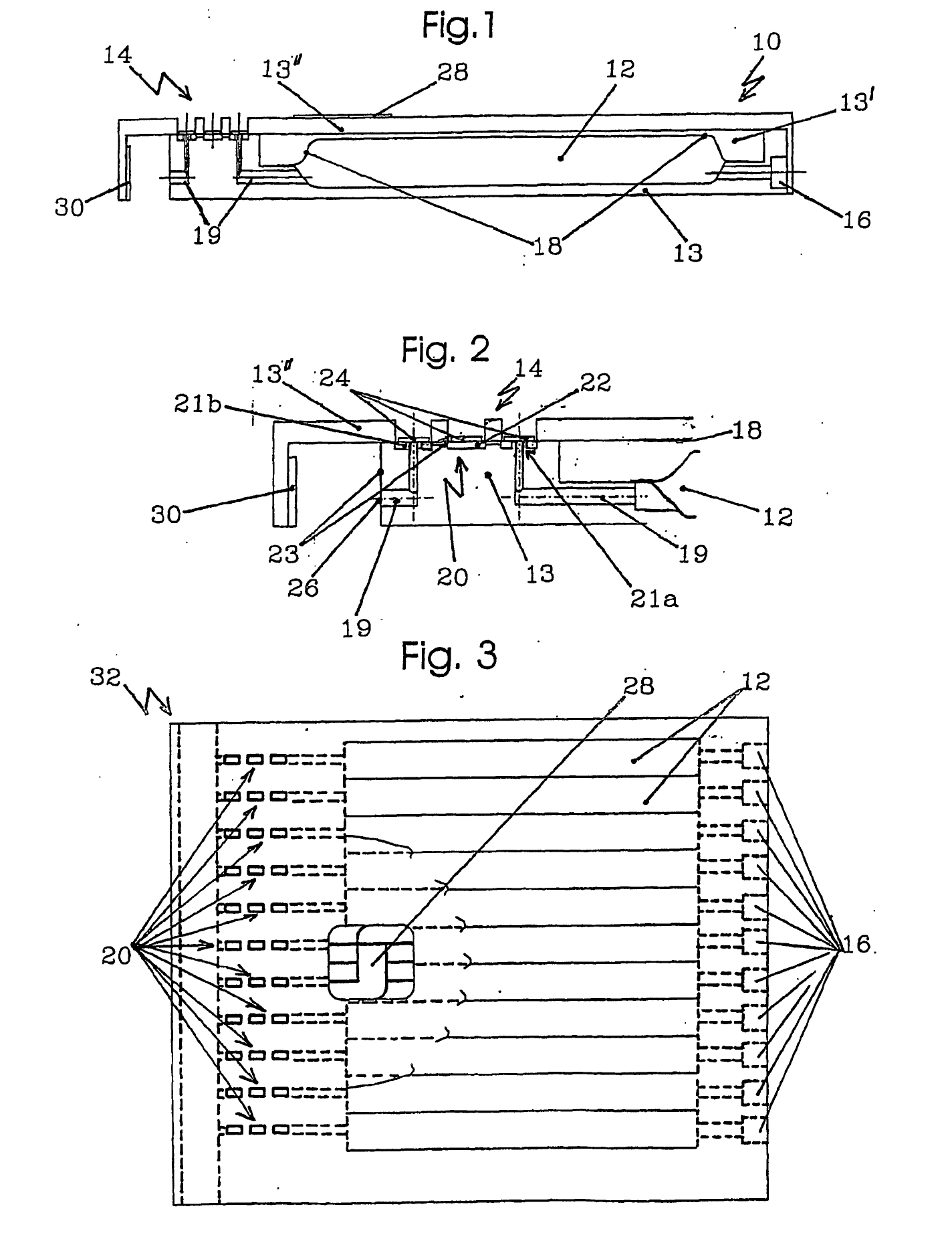

FIG. 1 is a section side view of the device in accordance with the invention for dispensing a scent;

FIG. 2 is a side view on a magnified scale of the dispenser belonging to the device as shown in FIG. 1;

FIG. 3 is a plan view of the device in accordance with the invention comprising a plurality of units for dispensing a variety of scents;

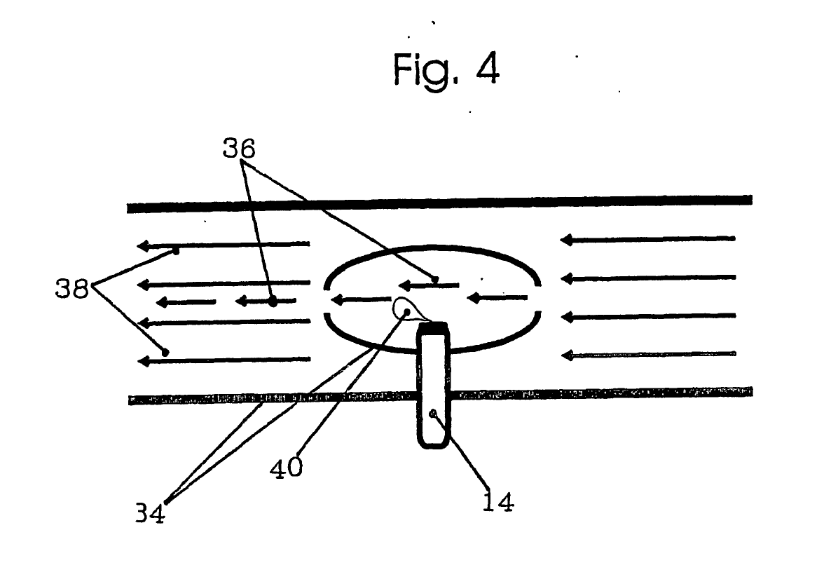

FIG. 4 is a diagrammatic illustration of a restrictor for generating an inner gas stream as a vehicle for a scent with an outer gas stream surrounding the inner gas stream.

Referring now to FIG. 1 there is illustrated on a magnified scale a device 10 for dispensing a scent including a housing 13, whose dimensions are roughly 40 mm×6 mm×6 mm. The device 10 comprises a tank 12, the bottom part of which is formed by the housing 13. The upper part of the tank 12 is formed by a membrane film 18 hemetically sealed to the edges of the tank bottom part. Formed at the rear end of the tank 12 is a filling port 16, by means of which the tank can be evacuated and filled with a scent. Evacuating the tank 12 via the filling port 16 causes the membrane film 18 to comes into contact with the bottom part of the tank in the housing 13. The membrane film 18 is selected so that it can come into contact with the non-deformable bottom part of the tank without wrinkling or forming voids. On subsequently filling the tank 12 via the filling port 16 the membrane film 18 is released from the bottom part of the tank and filling is continued until the desired filling level is attained, the membrane film 18 then, as shown, being able to bulge in a receiving space 13^{I} formed in the housing 13. The filling port 16 is subsequently plugged or closed off by a check valve (not shown). A cover or finger guard 13^{II} applied to the housing 13 protects the membrane film 18.

A dispenser 14 for the scent is connected via a passage 19 to the tank 12 in the housing 13. When evacuating the tank 12 via the filling port 16 the dispenser is hermetically sealed so that no environmental medium can gain access to the tank. Referring now to FIG. 2 there is illustrated in more detail how the dispenser 14 is configured as a micro-peristaltic pump 20 including in the passage 19 with an inlet valve 21a, an outlet valve 21b and a pumping chamber 22. Assigned to the two valves 21a, 21b and the pumping chamber 22 is an actuator membrane 23 extending over these elements. The inlet valve works such that the actuator membrane 23 closes or opens the feed from the tank 12 to the pumping chamber 22. Actuating the inlet valve is done with an actuator 24 arranged on the actuator membrane 23, the actuator 24 in this particular example embodiment being a piezoelement. Normally, i.e. when the piezoelement is deactivated, the inlet valve 21a is closed. When the piezoelement is activated the actuator membrane 23 bulges by the action of the piezoelement, resulting in scent being delivered via the inlet valve 21a into the pumping chamber 22.

The pumping chamber 22 is provided as a cavity in the housing 13, it being closed off on an upper open side by the actuator membrane 23. Arranged at the outer side of the actuator membrane 23 is a further actuator 24 as an electrically operated piezoelement. Actuating the piezoelement assigned to the pumping chamber 22 produces differences in pressure in the pumping chamber which deliver the scent into or from the pumping chamber 22. In other words, actuating this piezoelement causes the actuator membrane 23 to bulge outwardly from the pumping chamber, resulting in a pressure drop in the pumping chamber 22 by which scent is suctioned into the pumping chamber through the opened inlet valve 21a and with the outlet valve 21b closed. By contrast, when the actuator membrane 23 is bulged into the interior of the pumping chamber 22 the pressure in the pumping chamber increases causing the scent to be delivered from the pumping chamber 22 to a orifice 26 of the passage 19 with the inlet valve 21a closed and outlet valve 21b open.

Corresponding to the inlet valve 21a the outlet valve 21b comprises the actuator membrane 23 with an actuator 24 arranged thereon. The outlet valve 21b works like the inlet valve, i.e. the valve is closed when the actuation is deactivated. Actuation of the two valves 21a, 21b is clocked differingly so that the two valves do not open at the same time and the environmental medium cannot enter the tank 12.

In the embodiment as shown, the actuator membrane 23 extends from the two valves 21a, 21b and the pumping chamber 22 up to the orifice 26 of the passage 19. An optionally small opening can be configured in the actuator membrane 23 as the orifice 26 for dispensing the scent.

Arranged opposite the orifice 26 at an angled extension of the finger guard 1311 is an electrically heated hot plate 30. The hot plate 30 can be activated by a controller so that it is turned on or off as required. On emerging from the orifice 26 the scent comes into contact with the heated hot plate 30 resulting in the scent evaporating on the hot plate whilst enhancing convection or diffusion of the scent into the environmental medium.

The device 10 comprises a contacter 28 for connecting a controller (not shown) so that the dispenser 14 can be activated. The contacter 28 is a contacter known from prior art as used for example on chip cards. By means of the achieved, i.e. via the controller activated actuation of the dispenser 14 the scent can be dispensed at specific motion picture sequences, or in TV or other information as made available to the user.

Referring now to FIG. 3 there is illustrated a cartridge 32 comprising a plurality of units of tank 12 and micro-peristaltic pump 20, eleven such units being provided in the cartridge, all of which can be activated via a contacter 28. The features and details evident from the FIGS. 1 and 2 for the device 10 read the same for the cartridge 32 and thus a repeat description is omitted. Depending on the intensity and call for the individual scents the tank 12 can be filled to a differing degree with scent and the duration of scent dispensing can be individually controlled.

Referring now to FIG. 4 there is illustrated a method in which a scent 40 is dispensed from a dispenser 14 in a restrictor or jetting device 34. The restrictor or jetting device 34 is illustrated diagrammatically, with which preferably a restricted, possibly also an accelerated inner gas stream 36 is formed from a uniform stream of air and which is surrounded by an outer gas stream 38. Because of the difference in the flow velocity the inner gas stream 36 as the vehicle for the substance is surrounded by the outer gas stream 38 and insulated from the environmental medium. The scent in the inner gas stream can thus be transported over a greater distance by the inner gas stream with no olfactory appreciation, due to mixing of the inner gas stream with the environmental medium being prevented by the insulating outer gas stream.

### LIST OF REFERENCE NUMERALS

10 device for dispensing a scent
12 tank
13 housing
13^{I} receiving space
13^{II} finger guard
14 dispenser
16 filling port
18 membrane film
19 passage
20 micro-peristaltic pump
21a inlet valve
21b outlet valve
22 pumping chamber
23 actuator membrane
24 actuator (piezoelement)
25 orifice
28 contacter
30 hot plate
32 device (cartridge)
34 restrictor
36 inner gas stream
38 outer gas stream
40 scent

1-13. (canceled)

14. A device for dispensing a substance, particularly a scent or fragrancy, comprising a tank for filling with the substance and a dispenser assigned to said tank, comprising a micro-peristaltic pump for delivering the substance into an environmental medium, said tank and its dispenser being configured as a unit closed off from the environmental medium after each dispensing to substantially prevent contact of said environmental medium with said substance contained in said tank, and said micro-peristaltic pump comprising an inlet valve and an outlet valve and at least one pumping chamber, wherein one wall each of said valve and pumping chamber is formed by a deformable actuator membrane on which the actuator assigned to said inlet valve, said outlet valve and said at least one pumping chamber are arranged, and said tank comprising at least in part a deformable wall which comes into contact with a non-deforamble tank wall on evacuation.

15. The device as set forth in claim 14, wherein said tank comprises a closable filling port via which said tank can be evacuated and filled.

16. The device as set forth in claim 14, wherein said actuators are electrically activated piezo elements.

17. The device as set forth in claim 14, wherein said actuators are electrically activated and thermally operating actuators.

18. The device as set forth in claim 14, wherein said dispenser comprises a nozzle for nebulizing said substance to be dispensed.

19. The device as set forth in claim 14, wherein a contacter is provided by means of which said device for dispensing said substance can be connected to a controller for actuating said dispenser.

20. The device as set forth in claim 14, wherein said device comprises means for identifying said substance contained in said tank and means for identifying said cartridge respectively.

21. The device as set forth claim 14, wherein said dispenser comprises a hot plate arranged in the delivery path of said substance.

22. The device as set forth in claim 14, wherein a blower is provided which generates the air stream for delivering said dispensed substance.

23. The device as set forth in claim 22, wherein a restrictor or jetting device is provided which splits the air stream generated by said blower into an inner gas stream and an outer gas stream surrounding said inner gas stream differing in flow velocity, said substance being dispensed in said inner gas stream.

24. The device as set forth in claim 14, wherein a plurality of units each comprising a tank and dispenser are combined into a single cartridge for dispensing differing substances.

25. The device as set forth in claim 14, wherein the elements of said device coming into contact with said substance are made of a material such as, for example, polyetheretherketon (PEEK) which is immune to reacting with said substance.

26. A method for dispensing a substance, particularly a scent wherein said substance is dispensed in a first inner gas stream, said inner gas stream being surrounded by a second outer gas stream, said inner gas stream and said outer gas stream differing in flow velocity.
